(19) United States
(12) Patent Application Publication
(10) Pub. No.: US 2026/0354816 A1
(43) Pub. Date: Nov. 12, 2026

(54) Suturing System, Suturing Method and Use of a Suture Sheet

(71) Applicant: Andres Maria Freschi, Ciudad Autonoma de Buenos Aires (AR)

(72) Inventor: Andres María FRESCHI, Ciudad Autonoma de Buenos Aires (AR)

(21) Appl. No.: 19/200,879

(22) Filed: May 7, 2025

(51) Int. Cl. A61B 17/04; A61B 17/00; A61B 17/06

(52) U.S. Cl. CPC A61B 17/0493; A61B 17/06066; A61B 17/06166; A61B 2017/00747

(57) ABSTRACT

This invention refers to a suturing system comprising a needle, a thread, and a suture sheet. This reduces the adverse effects of applying the thread directly to the skin and subcutaneous tissue, also allowing the suture to be left in place for a longer period of time. In other embodiments, the suturing method and the use of the suture sheet are claimed.

### FIELD OF THE INVENTION

This invention refers to suturing elements and procedures. It is particularly related to sutures for facelifts.

### BACKGROUND

A facelift is a surgery where a cut, also called an incision, is made in front of the ear. The skin of the face is then lifted, facial muscles are tightened, and skin is repositioned. Excess skin and fat can be removed during the surgery. Finally, incisions are closed with sutures.

The main problem with this type of procedure during the postoperative period is the formation of hematomas. A hematoma is a collection of blood. In these surgical procedures, it occurs between the skin lifted during surgery and subcutaneous tissue.

According to scientific articles [1-6], the reported incidence rate can range from 0.6% to 14.2%. This can result in longer recovery times [3] or, more typically, may require the patient to return to the operating room for treatment of these hematomas. Hematomas should be drained to minimize negative consequences such as skin necrosis, pain, nerve compression, etc.

Typically, this type of hematomas would occur on one side of the face only, as it is very rare to occur on both sides.

The Auersvald brothers [3] developed a suturing method, which they called cutaneous hemostatic net, to prevent the formation of postoperative hematomas. The method consists in suturing the skin and the tissue below the skin (subcutaneous tissue) in such a way as to generate a “seal” in the space between them.

According to article [3], they were able to virtually eliminate the occurrence of complications due to hematoma formation during the postoperative period.

On the other hand, this method requires applying the suture directly to the skin, which can be visually disturbing. Additionally, if not removed in a timely manner, it can leave marks that take several weeks to fade, eventually leading to permanent skin discoloration.

For this reason, the hemostatic net should not be left in place for more than 2 or 3 days after surgery.

Therefore, the hemostatic net can be visually counterproductive and create permanent marks.

To this end, the inventor has decided to study what would happen if some type of device was placed between the exposed suture thread and the skin, achieving surprising results.

### BRIEF DESCRIPTION OF THE DRAWINGS

These drawings illustrate certain aspects of some of the embodiments of this invention and should not be used to limit or define the invention.

FIG. 1 is a side view of a suture on a patient using the suture sheet.

FIG. 2 is a second side view of a suture placed on a patient using the suture sheet.

### DETAILED DESCRIPTION

The term “polymeric suture material” is defined as any material that can form a polymer and is suitable for use as a suture thread.

The term “foam polymer material” is defined as any material that is a polymer wherein monomer bonding creates a polymer that encloses air-containing spaces without a predetermined shape.

The term “middle region” is defined as a region that is in the center of the suture sheet or does not extend to the edges of the suture sheet. This can range from 1 cm from the edge to the physical center of the suture sheet.

The term “system” can be used interchangeably to describe a system, kit, apparatus, or device.

In a first embodiment, the invention is a suturing system comprising a needle, a suture thread, and a suture sheet.

The needle is a ⅜″ Semicircular Needle.

The suture thread comprises any polymeric suture material suitable for this purpose. In a preferred embodiment, the suture thread is made of 5-0 nylon.

The suture sheet comprises any foam polymer material. In preferred embodiments, the foam polymer material is selected from the group consisting of high-density polyurethane (HDPE), low-density polyurethane (LDPE), some other type of polyurethane, or a combination thereof.

The suture sheet may have a thickness between 1 mm and 10 mm, preferably between 2 mm and 10 mm.

The shape of the suture sheet varies according to the shape of the body part it will rest on. Particularly, for facelifts, a piece of the suture sheet is removed to allow the ear to pass through the mid-region, depending on the size of the patient's ear. The edges are trimmed to cover the entire skin surface where the suture sheet will rest. Therefore, the shape of the suture sheet is irregular, although it can also be square, rectangular, circular, or some other shape.

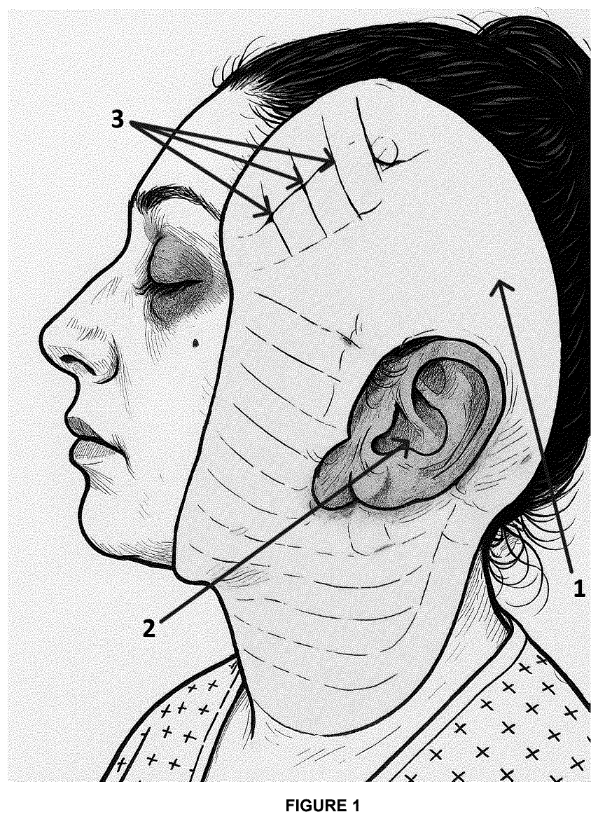

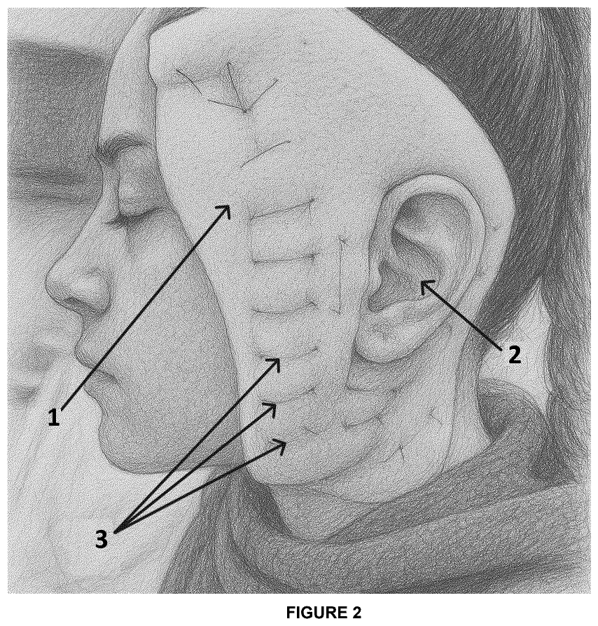

FIGS. 1 and 2 show an example of the application of the suture sheet to the sutures after a facelift surgery. The suture sheet (1) has an opening (2) through which the ear passes. Sutures (3) are applied to different areas of the suture sheet, firmly securing the suture sheet and distributing the applied tension.

Sutures (3) can be positioned at different points as seen in FIGS. 1 and 2. The shape of the suture sheet follows an irregular shape, but adapts to the surface where suture must be applied.

The invention is not only effective in facelift surgery but can also be used for the same purpose in any surgery where post-surgical compression of the “dead space” is required. For example, surgical treatment of gynecomastia, surgical correction of symmastia, etc.

In a second embodiment, the suturing method comprises placing the suture sheet on the skin to be sutured; passing, as a first step, the suture sheet placed on the skin with the needle and suture thread; passing, as a second step, the skin with the needle and suture thread; passing, as a third step, the subcutaneous tissue with the needle and suture thread; passing, as a fourth step, the subcutaneous tissue with the needle and suture thread in an opposite or substantially opposite direction to that of the third step; passing, as a fifth step, the skin with the needle and suture thread in an opposite o substantially opposite direction to that of the second step, and, passing, as a sixth step, the suture sheet placed on the skin with the needle and suture thread in a reverse or substantially reverse direction to that of the first step.

Particularly, the direction the needle and thread pass through is one of the following: perpendicular to the suture sheet, the skin, and subcutaneous tissue, or at an angle other than 90° to the suture sheet, the skin, and subcutaneous tissue.

In a third embodiment, the use of a suture sheet as a material to be placed on the skin during the suturing process is claimed as it reduces the tension caused by the suture thread on the skin and increases the time the patient can remain with the suture in place.

These three embodiments are technically suitable to create more moderate compression on the skin while distributing the tension of the thread throughout the foam. Thus, possible skin damage that may be created when applying the suture thread directly to the skin is reduced

Likewise, the number of needle passes can be reduced as compressive forces are distributed over the skin.

Additionally, these technical effects allow the suture to be left in place for a longer period of time, which is beneficial for patients prone to bleeding or hypertensive patients. Extending the time of application of this type of suture reduces the possibility of hematoma formation.

Moreover, the suture sheet reduces the negative impression that applying sutures directly to the skin often produces. It has been observed to generate greater patient acceptance.

### REFERENCES

[1] Baker D C, Stefani W A, Chiu ES. “Reducing the incidence of hematoma requiring surgical evacuation following male rhytidectomy: a 30-year review of 985 cases” Plast Reconstr Surg. 2005; 116(7): 1973-1985. doi: 10.1097/01.prs.0000191182.70617.e9
[2] Rees T D, Barone C M, Valauri F A, Ginsberg G D, Nolan W B. “Hematomas requiring surgical evacuation following face lift surgery” Plast Reconstr Surg. 1994; 93(6): 1185-1190. doi: 10.1097/00006534-199405000-00012
[3] Auersvald A, Auersvald L A. “Hemostatic net in rhytidoplasty: an efficient and safe method for preventing hematoma in 405 consecutive patients” Aesthetic Plast Surg. 2014; 38(1): 1-9. doi: 10.1007/s00266-013-0202-5
[4] Ramanadham S R, Mapula S, Costa C, Narasimhan K, Coleman J E, Rohrich R J. “Evolution of hypertension management in face lifting in 1089 patients: optimizing safety and outcomes” Plast Reconstr Surg. 2015; 135(4): 1037-1043. doi: 10.1097/PRS.0000000000001131
[5] Trussler A P, Hatef D A, Rohrich R J. “Management of hypertension in the facelift patient: results of a national consensus survey” Aesthet Surg J. 2011; 31(5): 493-500. doi: 10.1177/1090820X11411292
[6] Berner R E, Morain W D, Noe J M. “Postoperative hypertension as an etiological factor in hematoma after rhytidectomy. Prevention with chlorpromazine” Plast Reconstr Surg. 1976; 57(3): 314-319. doi: 10.1097/00006534-197603000-00006

What is claimed is:

1. A suturing system comprising:
a needle;
a suture thread; and
a suture sheet.

2. The suturing system of claim 1, wherein the needle is a ⅜ semi-circular needle.

3. The suturing system of claim 1, wherein the suture thread is made of 5-0 nylon.

4. The suturing system of claim 1, wherein the material of the suture sheet is selected from the group consisting of high-density polyurethane (HDPE), low-density polyurethane (LDPE), some other type of polyurethane, or a combination thereof.

5. The suturing system of claim 1, wherein thickness of the suture sheet is between 1 mm and 10 mm and preferably between 2 mm and 10 mm.

6. The suturing system of claim 1, wherein the suture sheet has an irregular shape and an opening for the ear to pass through.

7. A suturing method comprising:
placing the suture sheet on the skin to be sutured;
passing, as a first step, the suture sheet placed on the skin with the needle and suture thread;
passing, as a second step, the skin with the needle and suture thread;
passing, as a third step, the subcutaneous tissue with the needle and suture thread;
passing, as a fourth step, the subcutaneous tissue with the needle and suture thread in an opposite or substantially opposite direction to that of the third step;
passing, as a fifth step, the skin with the needle and suture thread in an opposite o substantially opposite direction to that of the second step; and,
passing, as a sixth step, the suture sheet placed on the skin with the needle and suture thread in a reverse or substantially reverse direction to that of the first step.

8. Use of a suture sheet, which is placed on the skin during the suturing process.
